(19) United States
(12) Patent Application Publication
(10) Pub. No.: US 2006/0039999 A1
(43) Pub. Date: Feb. 23, 2006

(54) Pharmaceutical composition for inhibition of tumor growth or metastasis

(75) Inventors: Jui Yi Lin, Taipei City (TW); Wendy Baowan Mach, Taipei City (TW); Po-Hsun Chen, Taipei County (TW)

Correspondence Address: BACON & THOMAS, PLLC, 625 SLATERS LANE, FOURTH FLOOR, ALEXANDRIA, VA, 22314, US

(73) Assignee: Hepaguard Biotechnology (Int'l) Co., Ltd., Taipei County (TW)

(21) Appl. No.: 11/156,522

(22) Filed: Jun. 21, 2005

(30) Foreign Application Priority Data
Aug. 17, 2004 (TW) 93124644

(51) Int. Cl. A61K 36/185

(52) U.S. Cl. 424/725

(57) ABSTRACT

A pharmaceutical composition for inhibition of tumor growth or metastasis which comprises an effective amount of Phyllanthus urinaria L. extracts, or the combination of the foregoing Phyllanthus urinaria L. extracts and pharmaceutical acceptable carries, adjuvants or excipients.

### BACKGROUND OF THE INVENTION

1. Field of the Invention

The invention is related to a pharmaceutical composition containing Phyllanthus urinaria L. extracts for inhibition of tumor growth or metastasis, which can be applied in tumor treatment.

2. Description of Prior Art

Tumors can be classified as benign and malignant in accordance with their basic characteristics, and malignant tumor is also known as cancer. The number of cancer deaths per year worldwide reaches about several millions. In Taiwan, cancer has ranked the first three of the ten leading causes of death. Many cancer patients are unaware of the basic facts about the disease and tend to ignore even the most simple of its signs and symptoms, which, if observed at an early stage, would help save thousands of lives. Therefore, for cancer patients, it is important to diagnose at early stage to improve cancer treatment. If metastasis does not occur, tumor can be removed by surgery, but if metastasis happens, cancer cells may adhere to blood vessels by blood viscosity, then penetrate the basement membrane surrounding the blood vessels and circulating in the bloodstream or the lymphatic system in the body, establishing tumor in a new tissue or organ of the body.

Once the metastasis occurs, resection cannot be performed, radiotherapy or chemotherapy is administered instead to kill creeping cancer cells. Immune system, sanguification, digestion system, liver, kidney, heart, and lung, however, may be poisoned thereby to cause reduction of leukocyte, bad appetite, hair loss, and decrease of immunity, and are doubtlessly harmful for cancer patients at high risk of tumor development. Therefore, metastasis often results in about 90% mortality rate and high recurrence rate in several years. Currently, cancer treatment including surgery resection, chemotherapy, and radiotherapy merely removes tumor on the specific sites, but cannot avoid metastasis completely. Accordingly, there is a pressing need to develop an anti-cancer drug which can resist metastasis and less harmful to the human body.

Phyllanthus urinaria L. is an annual or biennial herbaceous plant belonging to the genus Phyllanthus of Euphorbiaceae. The Phyllanthus plants distribute world widely and more than 600 different species have been catalogued thus far. The Height of Phyllanthus urinaria L. varies between 20 and 30 cm, short stem which is angular with numerous distiches, elliptic-oblong leaves. The flowering time is autumn, monoecious with staminate flowers and solitary pistillate flower borne axillary. It is found to distribute mainly in the wild, hillside and meadow etc. Phyllanthus urinaria L. is a traditional Chinese medicine prescription described in historical books. According to the description of Ben-Tsao-Gang-Mu, chapter Shi-yi, Phyllanthus urinaria L. treats numerous diseases in pediatrics, various malnutrition, emaciation, and possible loss of sight. Additionally, Phyllanthus urinaria L. has also been used to prevent from liver disorders and treat liver diseases for several hundred years. Based on recent pharmacologic studies, Phyllanthus urinaria L. is shown to hold liver protective benefits and be effective in preventing primary liver cancers. It is also shown that carbon tetrachloride (CCL₄) and galactosamine-induced liver injury can be protected by the extracts and sub-extracts of Phyllanthus urinaria L.

Reports regarding treating cancer with Phyllanthus plants, for example, Powis and Moore (1985), and Pettit et al. (1990) discovered glycosides purified from Phyllanthus acuminatus possess the anti-cancer effects in murine P-388 lymphocytes and B-16 melanin cell lines; Jeena et al. (1999), Rajeshkumar and Kuttan (2000) discovered Phyllanthus amarus can protect liver from N-nitrosodiethylamine-induced hepatoma in the animal experiments, Giridharan et al. (2002) discovered that 7′-hydroxy-3′,4′,5′,9,9′-pentamethoxy-3,4-methenyldioxylignin purified from Phyllanthus urinaria L. inhibits the activity of telomerase and expression of bcl2, as well as the activates apoptosis-inducers, caspase 3 and caspase 8, and is therefore considered applying in cancer treatment in the future. Furthermore, the Graduate Institute of Clinical Medical Sciences of Chang Gung University reported the aqueous phase extracts from Phyllanthus urinaria L. significantly inhibit the growth of lewis lung cancer cells in vitro in the journal of Life Sciences (2003), and the mechanism thereof is that Phyllanthus urinaria L. extract activates caspase 3 and inhibits the expression of bcl2. Accordingly, Phyllanthus urinaria L. certainly has great potential in tumor treatment.

Although Phyllanthus urinaria L. has great potential in tumor treatment, tests such as animal tests or clinical trials must also be performed to assure its clinical curative effects on tumor because experiment results of in vitro, cellular, and animal tests can vary unpredictably.

### SUMMARY OF THE INVENTION

In view of the curative potential of Phyllanthus urinaria L. on tumor, the present invention provides a pharmaceutical composition for inhibition of tumor growth or metastasis containing an effective amount of Phyllanthus urinaria L. extract. The foregoing pharmaceutical composition further comprises a pharmaceutical acceptable carrier, additive or adjuvant. The Phyllanthus urinaria L. extract is the initial extract obtained by extracting dry Phyllanthus urinaria L. with solvent. The pharmaceutical composition is suitable for inhibiting growth of various cancer cells or metastasis, for example, but not limited to rectum or liver cancer cells.

The Phyllanthus urinaria L. extract can be extracted from the Phyllanthus urinaria L.'s root, stalk, leaf, flower, fruit, seed, or combination thereof, and can be manufactured based on the needs into powders, granules, liquid, gel, or cream by the process that is known to one skilled in the art.

The pharmaceutical composition containing Phyllanthus urinaria L. extracts with an effective dosage can be provided in the form of food, drink, drug, reagent, or nutrient supplement, and can be administered via oral, injection, inhalation, hypodermis implantation, or transdermal administration.

The effective amount refers to an amount of the composition that is capable of producing a medically desirable result in a treated subject. Take tumor treatment as an example, compared to an untreated subject, the desirable result comprises the decrease of tumor mass, growth rate, metastasis, alleviation of symptom, extension of life, and/or improvement of life quality. The exact dosage for administration depends on the types, extent or symptom of the disease, as well as the health conditions, age, sex, weights, or drug toleration of the subject to be administered. The amount for administration also varies with the extent, severity, and type of tumor. One skilled in the art can decide the suitable dosage for administration according the foregoing or other factors.

The dosage of Phyllanthus urinaria L. extracts in an animal test (mouse test) provided by the invention is about 0.2˜1.8 g/kg/day, and curative effects are better as dosage increases, that is, dose-dependent. There are different methods for converting from the dosage of mouse to that of human such as, liver surface area conversion. According to the conversion, the dosage of 0.2˜1.8 g/kg/day for mouse is converted to about the dosage of 16.9˜152.3 mg/kg/day for human. An alternative way is dividing the dosage for mouse by coefficients 1˜100 set between mouse and human, so as to obtain the dosage for human, which is about 20˜1800 mg/kg/day. To determine a precise dosage, however, one skilled in the art should further consider the extent, severity, and type of tumor and the individual's physical constitution, such as health conditions, age, sex, weights, or drug tolerance. The effective dosage of Phyllanthus urinaria L. provided by the invention is, but not limited to, about 0.2˜3600 mg/kg/day. For example, for the subjects to be administered whose weights are among 10˜100 kg, a preferable dosage is about 2 mg˜360 g/day.

According to kinds of diseases or symptoms, the present pharmaceutical composition can be administered through various ways, such as, but not limited to, oral capsule, liquid suspension, and tablet or through parenteral administration. The parenteral administration includes, for example, systematically administration such as by intramuscular, intravenous, subcutaneous or intraperitoneal administration. The foregoing pharmaceutical composition can also be administered via oral (such as food content), topical injection, and inhalation (such as via bronchial, nasal, oral inhalation or nasal dropping, or via rectal administration.

The oral formulations of the pharmaceutical composition comprise solid medicaments, such as, but not limited to, capsule, tablet, sugar coated tablet, pill, powder, or granule, and solution, emulsion, suspension, syrup, or elixir (liquid), and can be further prepared with films such as enter-films by the conventional method. Additionally, the release time of active ingredients of the pharmaceutical composition can be controlled to remain or prolong the curative effects.

The Phyllanthus urinaria L. extracts can be also provided with pharmaceutically acceptable carriers, adjuvants, or excipients. The proper excipients comprises, but not limited to, lactose, manntiol, glucan, glucose, glutamate, gelatin, sorbitol, trehalose, sucrose, starch, microcrystalline cellulose, methylcellulose, Acacia gum, or combination thereof. The Phyllanthus urinaria L. extracts provided by the invention can be not only used alone but combined with adjuvants such as other anti-cancer drugs comprising, but not limited to, adriamycin, daunomycin, aclamycin A, dactinomycin D, mitomycin C, chromycin A3, vindesine, vinblastine, vincristine, pacilitaxel, 5-flurouridine, 5-deoxyuridine, methylzinum, tegafur, carmofur, cytosinearabinoside, cyclocytidine, 6-sulfhydrylpurine, cisplatin, kerastin, or estramstin. The invention provides the formulation such as solution, emulsion, or capsule in accordance with selected administration and different diseases or symptoms. Proper drug carrier could contain inert ingredients that will not substantially react with compounds of the present invention. Applicable drug formulation techniques may refer to methods described in Remington's Pharmaceutical Sciences, Mack Publishing Company, Easton, Pa. The carriers used in non-oral administrations comprise sterile water, normal saline, sterile saline (comprising about 0.9 mg/ml of phenyl alcohol), phosphate buffer saline solution, Hanks solution, Ringers lactose solution, or the like. Additionally, methods for packaging combinations into capsules such as covering hardened gelatin or cyclodextrin has been known to one skilled in the art. (Controlled Release of Biological Active Agents, John Wiley and Sons, 1986.) The “tumor”, here, means a disease, symptom, or disorder which cell's abnormal hyperplasia leads to, unnatural cell growth, reduction of differentiation, moreover, regional tissue invasion and metastasis. The major feature of tumor is hyperplasia of abnormal cells in normal tissue. Therefore, the hyperplasic abnormal cells may then intrude peripheral tissue, lymphatic or vessels and transfer to regional lymph gland and far regions (means for metastasis). According to clinical data and biological research, cancer is a multiple-steps disease. A mature tumor grows from mild proneoplastic variation and develops into tumor through some processes, for example, tumor grows from promalignant abnormal cells via hyperplasia and metaplasia, specifically via dysplasia (referred to Robbins and Angell (1976) Basic Pathology, 2d ED., W, B. Saunders Co., Philadelphia, 68˜79).

The pharmaceutical composition provided by the present invention can prevent or treat each kind of tumor formed in the above conditions. In summation, “tumor” in the present invention means any abnormal cell growth whether cells become tumor cells or not.

The method for extracting the Phyllanthus urinaria L. comprises, but not limited to, the following steps. First, dry Phyllanthus urinaria L. is extracted with solvent to obtain an extract solution or to remove the solvent to form powders of Phyllanthus urinaria L. extracts. Then the Phyllanthus urinaria L. extracts are purified to form concentrated Phyllanthus urinaria L. extracts.

A detailed description is given in the following with reference to the accompanying drawings.

### BRIEF DESCRIPTION OF THE DRAWINGS

FIG. 1 shows variations of average weight percentages of laboratory mice within 15 days after implanting rectum cancer cells thereinto.

FIG. 2 is a histogram showing metastasis rate of cancer of laboratory mice.

FIG. 3 is a histogram showing liver weights of laboratory mice.

FIG. 4 is a histogram showing spleen weights of laboratory mice.

### DETAILED DESCRIPTION OF THE INVENTION

Experimental Mice

Six-week BALB/c male mice were introduced from the National Laboratory Animal Center. Four mice were put in a cage, and eight mice were arranged in a group. The room temperature was controlled within 20˜24° C. The light cycle time was 12 hrs. The way of breeding was free breeding.

Cell Lines

Rectum cancer cell line (CT26) was cultured in an IMDM (Iscove's Modified Dulbecco's Medium) containing 10% fetal bovine serum at 37° C. in 5% CO₂ condition.

Drugs

1. Polysaccharide Krestin (PSK): A clinical drug for treating cancer, and manufactured by Sankyo Co., Ltd.
2. Somnotol: containing anesthetic drugs such as sodium pentobarbital, and manufactured by MTCPHAR Co., Canada.
3. Powders of dry Phyllanthus urinaria L. extracts: produced by extracted with water, concentrated with reducing pressure, and then freeze-dried.
   Experimental Steps

Referring to the Table 1, six-week BALB/c male mice were divided into six groups, and eight mice were arranged in a group. Mice were bred with water, PSK (0.312 g/kg), or various dosages of Phyllanthus urinaria L. extract (0.2, 0.6, or 1.8 g/kg) except normal breeding.

TABLE 1
group of laboratory mice

| Groups | Breeding | Implanting tumor |
|---|---|---|
| group | Water | Yes |
| Pseudo-Operation | Water | No |
| group | | |
| Contrast group | PSK (0.312 g/kg) | Yes |
| Experimental group 1 | Phyllanthus | Yes |
| | extracts (1.8 g/kg) | |
| Experimental group 2 | Phyllanthus | Yes |
| | extracts (0.6 g/kg) | |
| Experimental group 3 | Phyllanthus | Yes |
| | extracts (0.2 g/kg) | |

Eight-week mice were injected with 10 μl/g of Somnotol (6.5 mg/ml) comprising sodium pentobarbital in accordance with weights thereof. Rectum cancer cell lines CT-26 in a proper concentration were then implanted into the spleens via an intra-spleen implantation (2*10⁴ cells/per mouse), and sealing their wounds. After mice waked, they were replaced to the original cage and bred with foregoing substance continuously. Finally, mice were scarified in the 15^{th} day after implanting tumor. Metastasis of cancer cells and pathological change in various organs were recorded to compare curative effects of the substance.

Experimental Result

1. Measurement of Weight Variations of Laboratory Mice:

After rectum cancer cell lines CT-26 were implanted into the spleens, the weight of each mouse was measured in the current day, the 8^{th} day after implanting, and the 15^{th} day after implanting, respectively, to compare effects of substance and dosage on appetite of mice and initial toxicity response.

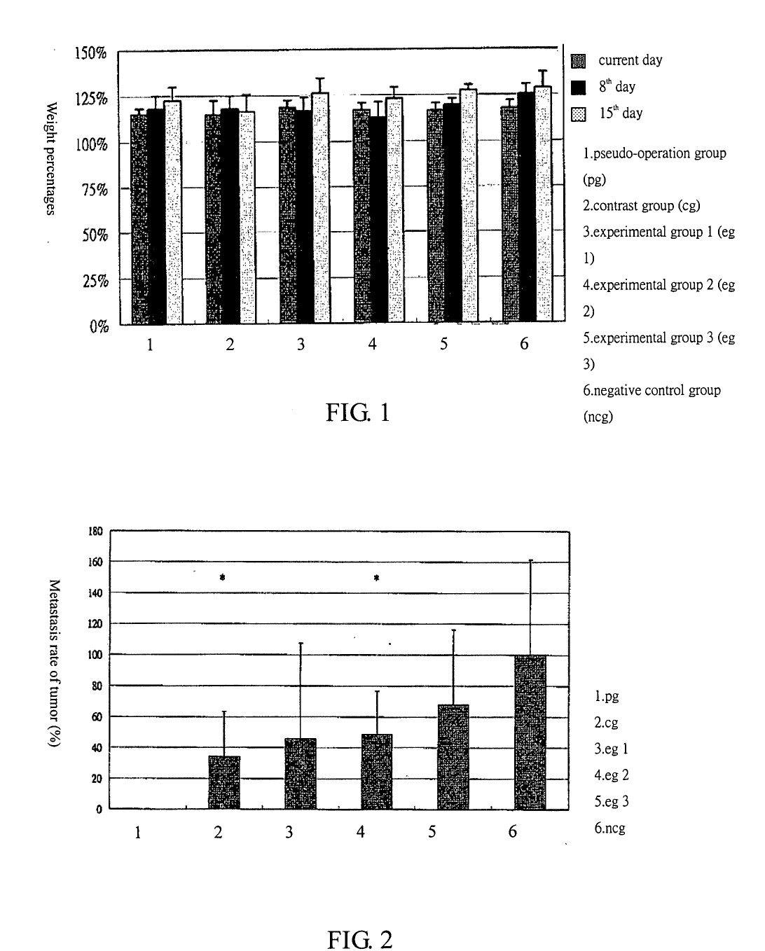

In the Table 2, the weight of each group is similar. Referring to FIG. 1 (histogram, converting from the data of the Table 2), the weight variation of the negative control group is the largest (average weight percentage of 128.8%), and the contrast group (administrating PSK) is the smallest (average weight percentage of 116.6%) within two weeks.

TABLE 2
variations of average weight percentages of the
groups

| | | | Average |
|---|---|---|---|
| | | Average | weight |
| | Average | weight | the 15^{th} |
| | weight | the 8^{th} | (after |
| | percentages | (after | implanting |
| | implanting | implanting | tumor) |
| Groups | tumor) (%) | tumor) (%) | (%) |
| Pseudo- | 115.10 ± 3.30 | 118.10 ± 7.00 | 122.80 ± 7.10 |
| group | | | |
| Contrast group | 114.70 ± 7.80 | 118.00 ± 7.10 | 116.60 ± 9.10 |
| Experimental | 118.60 ± 4.00 | 116.70 ± 7.40 | 126.30 ± 8.10 |
| group 1 | | | |
| Experimental | 117.10 ± 3.80 | 113.00 ± 8.50 | 123.30 ± 6.00 |
| group 2 | | | |
| Experimental | 116.60 ± 4.10 | 119.30 ± 3.60 | 127.50 ± 3.00 |
| group 3 | | | |
| group | 118.00 ± 4.00 | 125.50 ± 5.30 | 128.80 ± 8.50 |

2. Analysis of Tumor Metastasis Rate:

After sacrificed, the liver of each mouse was gilled and counted the tumor nodes on the surface thereof for comparing the variations of tumor metastasis rates.

The Table 3 (column 2) shows the percentage of tumor nodes in different groups comparing to those in the negative control group, for example, the experimental group 2 (breeding 0.6 g/kg Phyllanthus urinaria L. extracts) is 48.51±27.98%, the experimental group 3 (breeding 0.2 g/kg Phyllanthus urinaria L. extracts) is 67.78±48.23%, and the experimental group 1 (breeding 1.8 g/kg Phyllanthus urinaria L. extracts) is 45.77±61.67%, wherein the result of the experimental group 2 is significant. Additionally, one can recognize the variations among the groups more clearly by referring to the FIG. 2 (converting from the data of the Table 3).

TABLE 3
metastasis rates of tumor and livers and spleens

| | percentages | percentages | percentages |
|---|---|---|---|
| Groups | (%) | (%) | (%) |
| Pseudo- | 0 ± 0 | 6.38 ± 0.43 | 0.43 ± 0.06 |
| group | | | |
| Contrast group | 34.09 ± 29.5* | 7.19 ± 0.78 | 1.66 ± 0.8 |
| Experimental | 45.77 ± 61.67 | 7.15 ± 1.00 | 2.24 ± 0.65 |
| group 1 | | | |
| Experimental | 48.51 ± 27.98* | 6.99 ± 0.47* | 2.18 ± 0.73 |
| group 2 | | | |
| Experimental | 67.78 ± 48.23 | 7.61 ± 1.02 | 1.85 ± 1.05 |
| group 3 | | | |
| group | 100 ± 61.44 | 7.97 ± 1.25 | 2.31 ± 0.78 |

weight percentages of the groups (mean ± standard deviation, *significant (p < 0.05 by Student's t-test))

3. Analysis of Liver Weight:

measure the liver weight of each mouse and compare pharmaceutical effect of various drugs and dosage

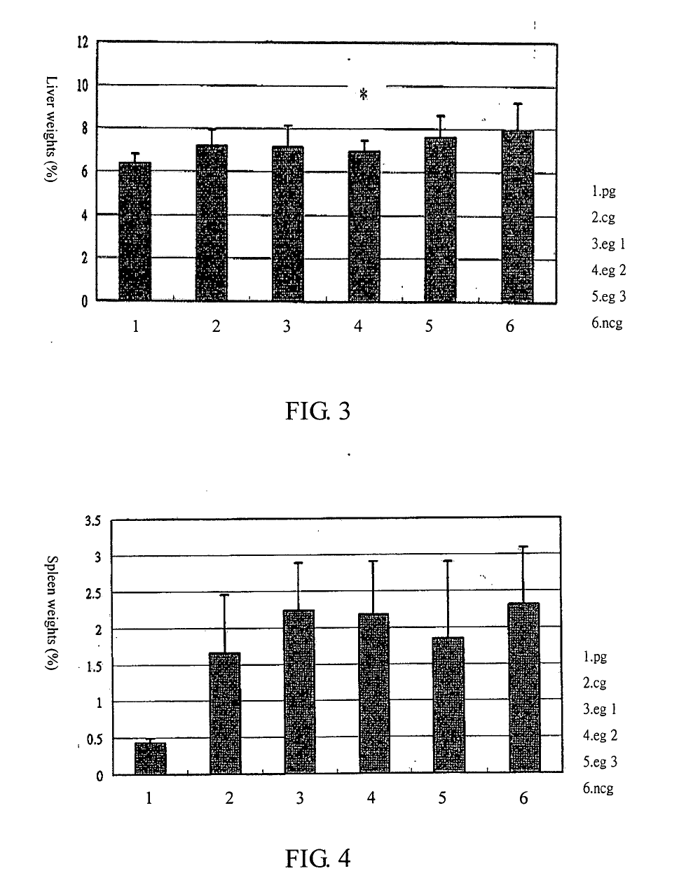

The Table 3 (column 3) shows the percentages of liver weight in body weight of different groups, for example, the experimental group 2 (breeding 0. 6 g/kg Phyllanthus urinaria L. extracts) is 6.99±0.47%, the experimental group 3 (breeding 0.2 g/kg Phyllanthus urinaria L. extracts) is 7.61±1.02%, and the experimental group 1 (breeding 1.8 g/kg Phyllanthus urinaria L. extracts) is 7.15±1.00%, wherein the result of the experimental group 2 shows the best curative effect. Clearly, Phyllanthus urinaria L. extracts can effectively inhibit liver gross. Additionally, one can recognize the variations among the groups more clearly by referring to the FIG. 3 (converting from the data of the Table 3).

4. Analysis of Spleen Weights:

measure the spleen weight of each mouse to compare various substances and dosages thereof in inhibiting the growth of transplantation tumor in-situ.

The Table 3 (column 4) shows the percentages of liver weight in body weight of different groups, for example, the experimental group 3 (breeding 0.2 g/kg Phyllanthus urinaria L. extracts) is 1.85±1.05%, the experimental group 2 (breeding 0.6 g/kg Phyllanthus urinaria L. extracts) is 2.18±0.73%, and the experimental group 1 (breeding 1.8 g/kg Phyllanthus urinaria L. extracts) is 2.24±0.65%, wherein the result of the experimental group 3 shows the best curative effect. Clearly, Phyllanthus urinaria L. extracts can effectively inhibit the growth of tumor implanted via an intra-spleen implantation. Additionally, one can recognize the variations among the groups more clearly by referring to the FIG. 4 (converting from the data of the Table 3).

The experimental results described above indicate that Phyllanthus urinaria L. extracts have a significant potency of anti-cancer growth and anti-metastasis, and can be used for, but not limited to, inhibiting growth and metastasis of rectum cancer cells. Therefore, the pharmaceutical composition containing Phyllanthus urinaria L. extracts with an effective dosage, pharmaceutical acceptable carriers, additives, or excipients for inhibition of cancer growth and metastasis provided by the invention is obviously useful to treat cancer.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. A pharmaceutical composition containing Phyllanthus urinaria L. extracts with an effective amount for inhibition of tumor growth or metastasis.

2. The pharmaceutical composition as claimed in claim 1, further comprising, pharmaceutical acceptable carrier, adjuvants, or excipients.

3. The pharmaceutical composition as claimed in claim 1, wherein the Phyllanthus urinaria L. extracts is obtained by extracting with solvent, condensing, and drying.

4. The pharmaceutical composition as claimed in claim 3, wherein the solvent is water.

5. The pharmaceutical composition as claimed in claim 1, wherein the Phyllanthus urinaria L. extracts is extracted from the Phyllanthus urinaria L.'s root, stalk, leaf, flower, fruit, seed, or combination thereof.

6. The pharmaceutical composition as claimed in claim 1, wherein the Phyllanthus urinaria L. extracts are shaped to powders, granules, liquid, gel, or cream.

7. The pharmaceutical composition as claimed in claim 1, wherein the Phyllanthus urinaria L. extracts are provided with food, drink, drug, reagent, or nutrient supplement.

8. The pharmaceutical composition as claimed in claim 1, wherein the Phyllanthus urinaria L. extracts are administrated to a subject via oral, injection, inhalation, hypodermis implantation, or transdermal administration.

9. The pharmaceutical composition as claimed in claim 1, wherein the effective amount of the Phyllanthus urinaria L. extracts is 0.2˜3600 mg/kg/day.

10. The pharmaceutical composition as claimed in claim 1, wherein a dosage per day of the Phyllanthus urinaria L. extracts is 2 mg˜360 g.

11. The pharmaceutical composition as claimed in claim 1, wherein the tumor is rectum cancer cells.

12. The pharmaceutical composition as claimed in claim 1, wherein the tumor is liver cancer cells.
